(19) United States
(12) Patent Application Publication
(10) Pub. No.: US 2004/0018991 A1
(43) Pub. Date: Jan. 29, 2004

(54) Topical treatment for mastalgia

(76) Inventors: Alfred Schmidt, Hamburg (DE); Heinrich Wieland, St. Peter (DE)

Correspondence Address: Dike Bronstein Roberts & Cushman, Intellectual Property Practice Group, Edwards & Angell, P O Box 9169, Boston, MA, 02209, US

(21) Appl. No.: 10/416,096

(22) PCT Filed: Oct. 30, 2001
(86) PCT No.: PCT/EP01/12536

(30) Foreign Application Priority Data
Nov. 8, 2000 (DE) 100-54-294.8

(51) Int. Cl. A61K 31/704; A61K 31/56

(52) U.S. Cl. 514/26; 514/170

(57) ABSTRACT

A composition for medicinal treatment by means of topical administration is described, which contains an aromatase inhibitor, in addition to conventional constituents of topical forms of administration. The active ingredient or the composition containing this active ingredient is especially suitable for the prophylaxis and for the treatment of mastalgia.

### DESCRIPTION

The present invention relates to a topical composition for the treatment of mastalgia or its symptoms.

Mastalgia (also called mastodynia) is a state of painful sensations of the breast widespread in women. Estimates assume that about two thirds of the working female population suffer from such breast pains. Their cyclical temporal occurrence can easily be associated with the menstrual cycle. They are very often part of the symptom complex “premenstrual syndrome”. The causes of the symptoms occurring appear complex and little understood hitherto by the expert world.

Accordingly, the traditional treatment processes for mastalgia are very different with regard to principle and action target (see Overview by P. A. Holland and C. A. Gatelay in “Drugs” 48(5), page 709-716 (1994), title: “Drug therapy of mastalgia. What are the options?”). The possibilities include, for example the use of analgesics or diuretics. On the other hand, natural products, such as night candle oil and vitamin B6 preparations are often used, but to which none or hardly any effects going beyond a placebo effect are ascribed. Other attempts follow systemic intervention in the metabolism, in particular by systemic administration of the prolactin inhibitor bromocriptin, of the gonadotropin inhibitor danazol and of the antioestrogen tamoxifen. These systemic administrations, particularly using the cytostatic agent tamoxifen, are however associated with considerable side-effects and in spite of the proven effect from the physician's side, is advised only in particularly persistent cases. To reduce the side-effects, a treatment control with the administration of low doses is indicated. Also, surgical intervention for local biopsy is in the wide spectrum of possible treatment. From the specialist side, there are however likewise strong objections against such surgical interventions.

The object of the present invention was therefore to improve the treatment possibilities of mastalgia.

The object is achieved by a composition for topical administration, wherein the composition contains an aromatase inhibitor, in addition to conventional constituents of topical forms of administration. The aromatase-inhibiting active ingredient is thus present in a composition to be administered locally and topically.

A further object of the invention consists in the use of such an aromatase inhibitor or a composition containing the latter for the prophylaxis and/or treatment for mastalgia by means of local topical administration. The use according to the invention is directed at eliminating the breast pain symptoms and includes the production of a pharmaceutical product.

Preferred embodiments of the invention are characterised in the sub-claims to claim 1. The invention can be used in particularly suitable manner for local topical administration to the breast region of the female breast, in particular the painful zones. Hence, a mastalgia-alleviating effect may be achieved in advantageous manner specifically and selectively, but without noticeably restricting the physiological equilibrium with the normal sex hormone-body metabolism in the untreated, that is far larger body region.

The treatment concept of the invention follows a completely novel concept and differs fundamentally from traditional treatment concepts. Whereas traditional treatments are based mainly on systemic medication systems or occasionally on operative-surgical measures and are tainted with severe side-effects or other disadvantages, the invention takes a different route and engages effectively and locally in the extragonadal synthesis paths of sex steroids. Because the administration takes place not systemically, but locally and topically and inhibits a central enzyme in extragonadal biosynthesis in the peripheral, treated body region, the treatment made available by the invention is very low in side-effects and well tolerated. Local topical administration is therefore very useful not only for treatment in the acute situation of pain sensation, but also as a prophylactic measure.

The mechanism of the alleviating effect according to the invention for mastalgia is not clear. Firstly, a primary effect may be assumed, which may be based on the production of a lack of oestrogens, such as oestradiol, oestrone and oestrol, available locally in the body (for example in the skin or the glands and integumentary appendages) due to the reduction in local oestrogen synthesis. Secondly, a secondary effect is also conceivable, possibly because breast lipocytes may shrink due to the lack of local oestrogens and hence build up less tension or pressure. Possibly primary and secondary effects also supplement one another and counteract the over-sensitivity typical of mastalgia. In any case a local effect in the female breast is the basis of the active mechanisms, since the active ingredient passes there directly due to the topical administration without deviation via the blood circulation.

The inhibitor of (cytochrome-p450)-aromatase used as active ingredient locally inhibits the extragonadal oestrogen formation. Known aromatase inhibitors can be divided broadly into such steroidal types, such non-steroidal types and others. According to the invention, individual or at the same time different aromataseinhibitor compounds may be used as active ingredients in a composition.

Examples of aromatase inhibitors include the following substances:

Steroidal aromatase inhibitors:

4-hydroxyandrost-4-ene-3,17-dione (formestan and lentaron),

6-methyleneandrostra-1,4-diene-3,17-dione (exemestan),

10-(2-propynyl)estr-4-ene-3,17-dione (MDL 18962)

7 alpha-substituted androstenedione derivatives

1,4,6-androstatriene-3,17-dione (ATD)

10-oxirane and 10-thiirane-substituted androgens

10-propargylestr-4-ene-3,17-dione

10-propargylestr-4-ene-3,17-propionates 10-(2-propynyl) derivative

13-retro-antiprogesterins

14 alpha-hydroxy-4-androstene-3,6,17-triones (14 alpha-OHAT)

16- or 19-substituted androst-4-enes

19-(cyclopropylamino)-androst-4-ene-3,17-dione

19-(ethyldithio)-androst-4-ene-3,17-diones (ORG 30958)

19-oxiranyl- and 19-thiiranyl steroids

19-thiomethyl- and 19-azido-androstenedione

1-methyl-androsta-1,4-diene-3,17-diones (atamestan)

2,2-dimethyl-4-hydroxy-4-androstene-3,17-dione

3 alpha-methoxyandrost-4-ene-6,17-diones

3 beta-hydroxyandrost-4-ene-6-one derivatives

3-deoxyandrogen-19-oxygenated derivatives of 3-oxo-17 beta-carboxamido steroids

4-(phenylthio)-4-androstene-3,17-dione

4-(thio-substituted)-4-androstene-3,17-dione

4-acetoxy-4-androstene-3,17-dione

4-aminoandrostenedione

4-androstene-3,6,17-trione

4-hydroxyandrostenedione (4-OHA, CGP 32349)

4-methoxy-4-androstene-3,17-dione

4-oxygenated androst-5-ene-17-ones and their 7-oxo derivatives

4-thio-substituted derivatives of 4-androstene-3,17-dione

4-thio-substituted-4-androstene-3,17-dione derivatives

5 alpha-dihydronorethindrone (a metabolite of norethindrone)

5 alpha-reduced C19 steroids

5 alpha-androstane-17-ones with or without a carbonyl function at C-3 and/or C-6

6 alpha, 7 alpha-cyclopropane derivatives of androst-4-ene

6 alpha-fluorotestosterone

6 beta-propynyl-substituted steroids

6,7-aziridinyl steroid and related compounds

6-alkyl analogues of delta 1,4,6-androgens

6-alkyl analogues of delta 4,6-androgens

6-alkyl- and 6-arylandrost-4-ene-3,17-diones

6-alkylandrost-4-ene-3,17-diones of 7 alpha- and 7 beta-arylaliphatic-substituted androst-4-ene-3,17-diones

6-alkylandrosta-4,6-diene-3,17-diones and their 1,4,6-triene analogues

6-alkyl-substituted androgens

6-phenylaliphatic-substituted C19 steroids with 1,4-diene, 4,6-diene or 1,4,6-triene structure

6-bromoandrostenedione

6-hydroximinoandrostenedione

6-methyleneandrosta-1,4-diene-3,17-dione (FCE 24304)

6-methyleneandrosta-1,4-diene-3,17-dione (FCE 24304)

6-phenylaliphatic-substituted androst-4-ene-3,17-diones

6-substituted androst-4-ene analogues

7 alpha-(4′-amino)phenylthio-4-androstene-3,17-dione

7 alpha-substituted androsta-1,4-diene-3,17-diones

7 alpha-substituted androstenediones

7 alpha-(4′-amino)phenylthio-4-androstene-3,17-dione

7 alpha-arylaliphatic androsta-1,4-diene-3,17-diones

7 alpha-substituted androstenediones

7 substituted 4,6-androstadiene-3,17-diones

7 substituted steroids

androst-4-ene-3,6-dione derivatives

androst-5-ene-7,17-dione 19-nor- and 5 beta, 6 beta-epoxy derivatives

A- or B-ring-substituted derivatives of androst-4-ene-3,6,17-trione

A-ring-bridged steroid

bromoacetoxy 4-androstene-3-ones

delta 1,4,6-androgens

delta 4,6-androgens

epimer 6-hydroperoxyandrostenediones

estr-4-ene-3,17-dione (MDL 18962),

estr-4-ene-3,6,17-triones

flavonoids

RU486

Non-steroidal aromatase inhibitors:

6-[(4-chlorophenyl) (1H-1,2,4-triazol-1-yl)-methyl]1-methyl-1H-benzotriazole (vorazol),

2,2′-[5-(1H-1,2,4-triazol-1-yl methyl)-1,3-phenylene]bis (2-methylproprionitrile) (arimidex),

4-[1-(cyanophenyl)-1-(1,2,4-triazolyl)methyl]benzonitrile (letrozol),

{4-(5,6,7,8-tetrahydro-imidazo-[1,5a]-pyridin-5-yl) benzonitrile monohydrochloride (fadrozol)

pyridoglutethimide (rogletimid),

aminogluthetimide

1,2-imidazolylmethylcyclopentanol derivatives

1-[(benzofuran-2-yl)phenylmethyl]-triazoles and tetrazoles

1-[benzofuran-2-yl)-phenylmethyl]-imidazoles (substituted)

1-(benzofuran-2-ylmethyl)imidazoles of N,N-disubstituted 5-aminopyrimidine derivatives

1-imidazolyl(alkyl)-substituted dihydroquinolines and tetrahydroquinolines

1-pentyl-3-(4-aminophenyl)pyrrolidine-2,5-dione

1-phenyl-3-azabicyclo[3.1.0]hexane-2,4-dione

1-phenyl-3-azabicyclo[3.1.0]hexane-2,4-dione and analogues

3-alkylated 3-(4-aminophenyl)piperidine-2,6-diones

3-cycloalkyl-substituted 3-(4-aminophenyl)piperidine-2,6-diones

3-ethyl-3-(4-pyridyl)piperidine-2,6- and 5-alkyl derivatives

3-ethyl-3-(4-pyridyl)piperidine-2,6-dione analogues

4-amino-4H-1,2,4-triazole derivatives

4-cyclohexylaniline

aminoglutethimide

benzimidazole and imidazole compounds

delta 1,4-bisnorcholadienic acid

delta 1-testolactone

imidazole derivatives of pyrrolidonic and piperidonic imidazolyl-1,3,5-triazines

MR 20492 and MR 20494 (two indolizinone derivatives) pyridyl-substituted indanones, indanes and tetralines

s-triazine derivatives SEF19

substituted pyridines

testololactone

Other aromatase inhibitors:

8-bromo-cyclic adenosine monophosphate

FR901537

hexamethylmelamine derivative (SAE9)

insulin-sensitised troglitazones and ketoconazoles

letrozole (CGS 20267)

mefloquin

MPV-2213ad

N-n-octanoylnornicotine and other nornicotine derivatives

Org 33201

R 76713 and R 76713

sesquiterpene lactones

SH 489

TAN-931

thyroid hormones

tobacalkaloid derivatives

YM511

With regard to the designations of these substances as well as their availability, see for example “Red List”, Editio Cantor, Aulendorf (DE), (1999).

Such aromatase inhibitors are known per se, mainly as systemically used active ingredients for medical-therapeutic treatment of breast cancer. In this context, reference is made to the overview article by A. M. H. Brodi in: “J. Steroid Biochem. Molec. Biol.”, volume 49, No. 4-6, pages 281-287 (1994), A. M. H. Brodi in “Biochemical Pharmacology”, volume 34, No. 18, pages 3213-3219 (1985) as well as P. E. Goss and K. M. E .H. Gwyn in: “Journal of Clinical Oncology”, volume 12, No. 11, pages 2460-2470 (1994). To determine aromatase inhibition and subsequent oestrogen reduction, reference is made to the further literature lists indicated in the said overview articles, see for example A. M. H. Brodi et al. in: “J. Steroid Biochem. Molec. Biol.”, volume 7, pages 787-793 (1976), and D. A. Marsh et al. in: “J. Med. Chem.”, volume 28, pages 788-795 (1985).

In WO publication 96/08231, aromatase inhibitors are used for combating hair loss. In a medicament for influencing hair growth, an androgen receptor antagonist is present in addition to the aromatase inhibitor. However, the promotion of hair growth is a fundamentally different problem from mastalgia, and an androgen receptor antagonist designated in the publication is not necessary within the framework of the present invention.

Special azole derivatives and their aromatase-inhibiting and antimycotic action are also described in EP-A-0 575 210.

U.S. Pat. No. 4,937,250 discloses special compounds with nitrile and imididazolyl and optionally further substituted toluene skeletal structure (alphaheterocycle-substituted toluonitriles) as aromatase-inhibiting active ingredients and proposes the particular pharmaceutical compositions containing active ingredients for oral, rectal, transdermal or parenteral administration for oestrogen synthesisdependent states, such as gynecomastia, mammary and endometrial tumours, endometriosis and early pains.

U.S. Pat. No. 4,895,715 proposes a treatment for gynecomastia in the male, especially for a benign form of hypertrophy of the prostate, in which an antiandrogen (for example flutamid) is administered together with an aromatase inhibitor (4-hydroxyandrosterone) or with an antioestrogen (tamoxifen) orally or parenterally, that is systemically.

For further special applications, aromatase inhibitors have been described as substances which have a favourable effect for the cosmetic treatment of disturbed subcutaneous connective fatty tissue, in particular cellulite (see WO publication 97/36570), and for tightening and contracting fat cell-containing body parts, in particular breast tightening (see WO publication 99/17712). However, no conclusions may be drawn from such applications for the problem of treating mastalgia. A mastalgia-alleviating effect was not to be expected.

A novel substance selection first disclosed in WO publication 97/36570 within the class of aromatase inhibitors, namely of those aromatase-inhibiting sterols (glycines), which originate from soya (soya glycines, INCI name according to the Linné system), may also be used within the framework of the present invention. In this respect reference is made to WO publication 97/36570. This also applies to the oxidatively treated soya glycines likewise described there.

In combination with the aromatase inhibitors, a substance belonging to the group of anti-oestrogens may be included in the topical composition of the invention in order to increase the mastalgia-alleviating effect. The non-steroidal oestrogen antagonists tamoxifen (Z-2-[4-(1,2-diphenyl-1-butenyl)-phenoxy]-N,N-dimethylamine) and aminoglutethimide (3-(4-aminophenyl)-3-ethyl-2,6-piperidinedione) as well as their analogues and derivatives, for example 3-hydroxytamoxifen, 4-hydroxytamoxifen as well as 7 a-alkyl-sulphinyl-tamoxifen analogues (ICI 182,780) in particular should be mentioned as examples of substances of the anti-oestrogen class. This combination composition of the invention is advantageous due to the better tolerance of topical administration in contrast to systemic administration of, for example tamoxifen, with the side-effects associated therewith. One or more aromatase inhibitors may also be combined with one or more anti-oestrogens. The quantity ratio in terms of weight of aromatase inhibitor to anti-oestrogen to be used in the combination lies suitably in a range from 90/10 to 10/90, in particular in a range from 60/40 to 40/60.

In order to facilitate the therapeutic attempt of the present invention in more specific manner and to enable coordination, one or more of the aromatase inhibitors described above and optionally antioestrogens may be combined such that, with a further active principle in addition or at the same time, the formation and/or the action of dihydrotestosterone is inhibited. A particular advantage of the present invention is proven in that some of the above-mentioned steroidal aromatase inhibitors have the required effect of simultaneous inhibition of the enzyme 5-alpha-reductase, which is responsible for the reduction of testosterone to the active androgen dihydrotestosterone (DHT). Without the simultaneous inhibition of 5-alpha-reductase, after inhibition solely of the aromatase in the breast and in the breast skin, androgens predominate, which could lead to local masculinisation phenomena, such as hair growth or acne. Inhibition of the 5-alpha-reductase may be achieved not only by using such substances with simultaneous aromatase-inhibiting and 5-alpha-reductase-inhibiting action, but also by combined topical administration of an aromatase inhibitor without the inhibiting effect on 5-alpha-reductase and an inhibitor of 5-alpha-reductase.

Examples of 5-alpha-reductase inhibitors include the following, wherein classification is carried out according to type:

Type 1 Inhibitors:

LY191704 (benzoquinolinone)

4,7 beta-dimethyl-4-azacholestane-3-one (MK-386) and related 4-azasteroids

benzo[c]quinolizin-3-one

Type 2 Inhibitors:

benzophenone and indole carboxylic acids

N-tert-butyl-3-oxo-4-aza-5α-androst-1-ene-17-β-carboxamide

(finasterid)

Dual Inhibitors (Type 1 and Type 2):

3-carboxy-20-keto-steroids

6-azasteroid

4-aza-3-oxo-5 alpha-androst-1-ene-17 beta-N-aryl-6-azasteroids

FK143

Non-Steroidal Inhibitors:

4-(1-benzoylindol-3-yl) butyric acids

4-[3-[3-[bis(4-isobutylphenyl)methylamino]benzoyl]-1H-indol-1-yl]butyric acid benzanilide derivatives

carbamoylalkenyl)phenyloxy carboxylic acid derivatives

ethyl-4-(1-methyl-2-oxopiperid-5-yl) benzoate

FK143

N,N-bis(1-methylethyl)-4-[3-(1,2-dihydro-1-methyl-2-oxopyrid-5-yl)propyl]benzamide

phenoxybenzoic acid derivatives

carboxamide-substituted and phenylalkyl-substituted pyridones and piperidones sodium 4-[2-(2,3-dimethyl-4-[1-(4-isobutylphenylethoxy]benzeneamino)phenoxy]butyrate (ONO-3805)

(Z)-4-2-[[3-[1-(4,4′-difluorobenzhydryl)indol-5-yl]-2-pentenoyl]-amino]phenoxy] butyric acid (KF20405)

Steroidal Inhibitors:

17 beta-(N,N-diisopropylcarbamoyl)estra-1,3,5(10)-triene 3-sulphonic acid

17 beta-carbamoyl-1,3-5(10)-estratriene 3-carboxylic acids

17 beta-N,N-diethylcarbamoyl-4-methyl-4-aza-5 alpha-androstane-3-one (4-MA)

17 beta-N-(2-methyl-2-propyl)-carbamoyl-androst-3,5-diene 3-carboxylic acid

3-androstene-3-carboxylic acids (steroidal acrylates),

3-carboxy-17 beta-substituted steroid

4-aza-3-oxo-steroid family

4-hydroxy-androstenedione

4-methyl-4-aza-5 alpha-pregnan-3-one-20(S) carboxylate

6-methylene progesterone, 6-methylene androstene and 6-methylene androstane

derivatives

finasterid

progesterone

sodium 4-methyl-3-oxo-4-aza-5 alpha-pregnan-20 (S) carboxylate

steroidal A-ring arylcarboxylic acids

TZP-4238 (steroidal antiandrogen)

These substances for inhibiting the formation and/or action of dihydrotestosterone are also known per se, but only for the treatment of benign prostate hyperplasia (see “Red List”, Editio Cantor, Aulendorf (DE), (1999)).

The sterol 4-hydroxyandrostenedione as well as derivatives thereof, for example the above-mentioned formestan, which is very similar to androstenedione, or the soya sterols, should be mentioned as an example of substances having bifunctional ability, that is which may develop both an aromatase-inhibiting and a 5-alpha-reductase-inhibiting action.

In the sense of increased efficiency, those aromatase inhibitors and optionally those antioestrogen and/or 5-alpha-reductase inhibitors are preferred according to the invention which have a lipophilic character. Such lipophilic substances, particularly when they are introduced into a suitable pharmacological skin-permeation vehicle system, may easily pass through the skin layers as well as cell membranes and thus rapidly and efficiently unfold their effect. Such substances or the compositions containing these substances are therefore particularly well suited to topical administration. This applies particularly to the steroidal aromatase inhibitors, which due to the steroid structure naturally have a lipophilic character and usually have good to very good percutaneous ability for resorption and cell-membrane permeability. In addition, the lipophilic substances may be easily accumulated in the fatty tissue. Substances which do not naturally have required high lipophilia may be rendered lipophilic in a manner known per se by modifying or derivatising using lipophilic groups, without losing the mastalgia-alleviating effect.

If in individual cases percutaneous resorption causes problems, or if increased percutaneous resorption is to be achieved, agents for promoting percutaneous resorption may preferably be additionally employed in the composition used. Such agents for promoting percutaneous resorption are known. Hyaluronidates, dimethyl sulphoxide (DMSO) and the like are suitable examples for this.

For topical administration, a formulation of the material to be used suitable therefor may be selected, for example an ointment, a cream, a gel, an emulsion (lotio), a powder, an oil etc. For this purpose, the composition includes additives which are conventional for the corresponding formulation as ointment, cream, gel, emulsion, powder or oil etc. Traditional skin care agents which have been described as well as those which are commercially available are suitable in the particular formulations for use in the present invention. For example vegetable oils, such as almond oil, olive oil, peach-kernel oil, peanut oil, castor oil and the like, plant extracts, essential oils, vitamin oils, fats and materials similar to fats, lipoids, phosphatides, hydrocarbons, such as paraffins, Vaseline, lanolin, waxes and the like, detergents, further skin active ingredients, such as lecithin, wool alcohols, carotene and the like, skin nutrients, perfumes, cosmetic materials, alcohols, glycerine, glycols, urea, talc, preservatives, sunscreen agents, dyestuffs, such as titanium white and zinc white, antioxidants etc., serve as conventional additives for such formulations. Water generally serves as base substance. An O/W or W/O emulsion may be obtained with addition of emulsifiers, such as fatty alcohol sulphates, alkali metal soaps, lecithins, triethanolamine and the like.

So-called transdermal therapeutic systems (TTS) can also be used very well for the skin, in which the active ingredient may be administered to the skin via an adhesive carrier system, for example a plaster, continuously over a longer period of time in suitable doses.

The aromatase inhibitor may be used in the composition in a quantity that the mastalgia symptoms are effectively controlled or alleviated. This may also be determined as a function of the degree of severity of the mastalgia and matched to the particular application. For example, an active ingredient content in the total composition of 0.0001 to 10 weight percent (wt. %), preferably 0.001 to 5 wt. % and in particular 0.3 to 2 wt. %, is suitable. Appropriate quantity ranges are likewise suitable for the possible antioestrogens and/or 5-alpha-reductase inhibitors if required.

The content of the resorption-promoting agent optionally to be used depends primarily on the type of resorption-promoting agent. The particular content values traditionally used are thus completely suitable. Hyaluronidates for example may be used in a concentration of 0.01 to 1 wt. %, in particular 0.05 to 0.2 wt. %. A further content range, for example 1 to 25 wt. %, in particular 5 to 10 wt. %, is suitable for DMSO.

The further additives, which are optionally present, may be used in the quantities which are conventional for the particular formulations.

Examples for further illustration of the invention, which are not to be understood as limiting, are described below.

Treatments acting with 4-hydroxy-androstenedione for mastalgia syndrome are described, wherein instead of 4-hydroxy-androstenedione, other aromatase inhibitors may be used.

### EXAMPLE 1

I. L. female 17 years

Findings: Tension-related pains in both breasts occurring 3 to 5 days before each menstruation, apparently bundled shooting into the breast nipples. Then slowly decreasing pains, symptoms and signs and tension states during the menstruation period until about the 10^{th} day after onset of menstruation.

A cream treated with 0.5 wt. % of 4-hydroxy-androstenedione was used (0.5% of 4OH-androstenedione introduced into 100 ml of ash-based cream). 4 g were administered daily, and specifically 2 g applied externally to each breast and carefully massaged in.

Start of healing test about 3 weeks before the period.

Result of the first subsequent period:

Significantly reduced tension pain; symptoms and signs subsided more rapidly.

Pain-free just 5 days after start of menstruation.

Continuation of the healing test with daily administration of 4-OH-androstenedione cream 0.5% strength.

Result of the second subsequent period: complete freedom from pain

### EXAMPLE 2

M. D., female 34 years

Findings: For years, severe swelling and tension feeling occurring in each case 3-5 days before menstruation with diffuse pain symptoms and signs radiating into the thorax, which is almost unbearable until the onset of menstruation. For years, unable to work 1-3 days per month due to these pains. Disappearance of symptoms and signs only on 8^{th}-12^{th} day after onset of menstruation.

Healing test using 1% strength 4-OH-androstenedione cream (1 g of 4-OH-androstenedione introduced into 100 ml of ash-based cream).

Dose 1×daily 4 g, in each case 2 g applied to each breast.

The start of the healing test took place about 2 weeks before onset of menstruation.

Subsequent period 1: somewhat blander course; 1 day unable to work.

Subsequent period 2: Significantly lower swelling and tension feeling; considerably lower pain symptoms and signs; still only localised in breasts, no inability to work.

Continuation of the healing test using 0.5% strength 4-OH-androstenedione cream (0.5 g of 4-OH-androstenedione introduced into 100 ml of ash-based cream) 4 g daily, that is 2 g applied to each breast.

Subsequent periods: Hardly any swelling and tension feelings; practically pain-free. Since then, no mastopathy-related inability to work.

1. Composition for medicinal treatment by means of topical administration, characterised in that the composition contains an aromatase inhibitor, in addition to conventional constituents of topical forms of administration.

2. Composition according to claim 1, characterised in that the aromatase inhibitor has lipophilic character.

3. Composition according to claim 1 or 2, characterised in that the aromatase inhibitor is a steroid compound.

4. Composition according to claim 3, characterised in that the aromatase inhibitor is an aromatase-inhibiting sterol.

5. Composition according to claim 4, characterised in that the aromatase-inhibiting sterol originates from soya glycines.

6. Composition according to one of the preceding claims, characterised in that the aromatase inhibitor is present in a composition in a quantity of 0.001 to 5 wt. %, based on the total quantity of the composition.

7. Composition according to one of the preceding claims, characterised in that it also contains an anti-oestrogen.

8. Composition according to one of the preceding claims, characterised in that a substance which simultaneously has an inhibiting effect with respect to 5-alpha-reductase serves as aromatase inhibitor.

9. Composition according to one of claims 1 to 7, characterised in that it also contains a substance, which is different from the aromatase inhibitor used, which has a 5-alpha-reductase-inhibiting effect.

10. Composition according to one of the preceding claims, characterised in that it also includes agents for promoting percutaneous resorption.

11. Composition according to one of the preceding claims, characterised in that it is formulated as ointment, cream, gel, oil or emulsion or lotio.

12. Composition according to claim 11, characterised in that it includes additives which are conventional for the corresponding formulation as ointment, cream, gel, oil, emulsion or lotio.

13. Composition according to one of the preceding claims, characterised in that it is integrated in a transdermal system.

14. Use of an aromatase inhibitor or a composition containing an aromatase inhibitor for the prophylaxis and/or treatment of mastalgia by means of local topical administration.

15. Use according to claim 13, wherein a composition as defined in one of claims 2 to 13 is administered.
